(19) United States
(12) Patent Application Publication
(10) Pub. No.: US 2017/0143987 A1
(43) Pub. Date: May 25, 2017

(54) LASER-ASSISTED DRUG DELIVERY SYSTEM

(71) Applicant: THE CHARLES STARK DRAPER LABORATORY, INC., Cambridge, MA (US)

(72) Inventors: Noel Elman, Cambridge, MA (US); David Veysset, Cambridge, MA (US); Keith Nelson, Cambridge, MA (US)

(21) Appl. No.: 15/360,193

(22) Filed: Nov. 23, 2016

Related U.S. Application Data
(60) Provisional application No. 62/259,342, filed on Nov. 24, 2015.

(51) Int. Cl. A61N 5/06; A61K 9/14; A61K 41/00; A61M 5/00

(52) U.S. Cl. CPC A61N 5/062; A61M 5/007; A61K 9/14; A61K 41/00; A61N 2005/067

(57) ABSTRACT

According to various aspects and embodiments, a system and method for treating a target condition are provided. The treatment system may comprise a launch platform, a light source, and a controller coupled to the light source. The launch platform may include a substrate, a layer of absorption material, and a layer of microparticles comprising at least one therapeutic agent. The microparticles may be launched from the launch platform using light energy emitted from the light source and directed to a target condition for purposes of delivering a therapeutic agent to the target condition.

### CROSS-REFERENCE TO RELATED APPLICATIONS

This application claims priority under 35 U.S.C. §119(e) to U.S. Provisional Application Ser. No. 62/259,342 titled “LASER-ASSISTED DRUG DELIVERY (LADS) SYSTEM FOR INOPERABLE TUMORS,” filed Nov. 24, 2015, which is incorporated herein by reference in its entirety.

### BACKGROUND

Cancer is a leading cause of death worldwide. Cancer is a generic term for a large group of diseases that can affect any part of the body and is typically characterized by out-of-control cell growth. Cancer harms the body when altered cells divide uncontrollably to form lumps or masses of tissue called tumors. Tumors can grow and interfere with the digestive, nervous, and circulatory systems and they can release hormones that alter body function.

Current modalities to treat solid tumors rely on resection through invasive surgical procedures that are sometimes combined with pharmacological and radiological therapies. Life-threatening conditions that involve gliomas, glioblastoma multiforme (GBM), meningiomas, or pancreatic tumors require aggressive treatments that do not always result in improved patient outcome.

One of the primary reasons for such limited prognoses is that solid tumors may not be fully resected in spite of clear visualization and location using imaging techniques, such as MRI or CT. Solid tumors may result in inoperability due to difficult anatomical access, or because surgery would lead to compromised physiological functions. For example, pancreatic cancer is one of the most challenging malignancies, since the majority of patients have advanced disease on presentation. Furthermore, pancreatic tumors may not be fully resected when they have metastasized to a distal location, such as the liver, and gliomas may not be fully resected because of their location at critical cognitive loci. Inoperable pancreatic adenocarcinoma is a dilemma that oncologists frequently encounter. Only 15-20% of patients are diagnosed when cancer of the pancreas is still surgically resectable. Guidelines established by the National Comprehensive Cancer Network (NCCN) classify pancreatic tumors into three categories: resectable, unresectable, and borderline resectable tumors. Despite extensive investigations aimed at improving surgery, radiation, and systemic therapy for this disease, little progress has been made in recent years to improve overall mortality.

### SUMMARY

Aspects and embodiments are directed to systems and methods for treating a target condition. In accordance with one aspect of the invention, a system for treating a target condition is provided comprising a launch platform, a light source, and a controller coupled to the light source and configured to control light energy emitted from the light source. The launch platform may comprise a substrate, a layer of light absorption material deposited on the substrate, and a layer of microparticles comprising at least one therapeutic agent deposited on the layer of absorption material.

According to one embodiment, the layer of microparticles is arranged as a self-assembled monolayer. According to another embodiment, the substrate is transparent to infrared light. According to another embodiment, the substrate further comprises a layer of metal deposited on the substrate between the substrate and the layer of light absorption material.

According to one embodiment, the at least one therapeutic agent is a pharmaceutical or a radioactive material. According to another embodiment, the microparticles further comprise at least one of a metal, an imaging agent, a polymer, and a drug carrier.

According to another embodiment, the light source is a laser light source. According to another embodiment, the controller is configured to control at least one of the laser pulse energy and pulse duration.

According to some embodiments, the launch platform is configured to be interchangeable.

In accordance with another aspect of the invention, a device for treating a target condition is providing that comprises a housing, a launch platform positioned within the housing, the launch pad comprising a substrate, a layer of metal deposited on a first surface of the substrate, a layer of light absorption material deposited on the layer of metal, and a layer of microparticles comprising at least one therapeutic agent deposited on the layer of light absorption material. The device also comprises a light source coupled to the housing and configured such that light energy emitted from the light source is in communication with a second surface of the substrate.

According to one embodiment, the device further comprises a focusing lens disposed between the light source and the second surface of the substrate. The focusing lens may be configured to focus light energy passing therethrough.

According to another embodiment, the light source is configured as a laser light source coupled to an optical fiber such that a first end of the optical fiber is coupled to the laser light source and a second end of the optical fiber terminates in proximity to the focusing lens.

According to some embodiments, the device further comprises a controller in electrical communication with the light source and configured to control the light energy emitted from the light source. According to another embodiment, the launch platform is configured to be connected to the housing.

According to another embodiment, the at least one therapeutic agent is a pharmaceutical or a radioactive material. According to some embodiments, the layer of metal is gold and the layer of absorption material is PDMS.

In accordance with another aspect of the invention a method for forming a treatment device is provided comprising depositing a layer of metal onto a first surface of a substrate, depositing a layer of light absorption material onto the layer of metal, depositing a layer of microparticles comprising at least one therapeutic agent onto the layer of light absorption material, and coupling a light source to a second surface of the substrate.

According to one embodiment, the microparticles are deposited such that they form a self-assembled monolayer. According to another embodiment, the method further comprises providing a controller that is configured to control light energy emitted from the light source. According to another embodiment, the method further comprises positioning a focusing lens in between the light source and the second surface of the substrate.

Still other aspects, embodiments, and advantages of these example aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Embodiments disclosed herein may be combined with other embodiments, and references to “an embodiment,” “an example,” “some embodiments,” “some examples,” “an alternate embodiment,” “various embodiments,” “one embodiment,” “at least one embodiment,” “this and other embodiments,” “certain embodiments,” or the like are not necessarily mutually exclusive and are intended to indicate that a particular feature, structure, or characteristic described may be included in at least one embodiment. The appearances of such terms herein are not necessarily all referring to the same embodiment.

### BRIEF DESCRIPTION OF DRAWINGS

Various aspects of at least one embodiment are discussed below with reference to the accompanying figures, which are not intended to be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or nearly identical component that is illustrated in various figures is represented by a like numeral. For purposes of clarity, not every component may be labeled in every figure. In the figures:

FIG. 1 is a schematic of a treatment system in accordance with one or more aspects of the invention;

FIG. 2 is a cross-sectional view of a launch platform in accordance with one or more aspects of the invention;

FIG. 3A is a schematic of a treatment device being used to treat a tumor in accordance with one or more aspects of the invention;

FIG. 3B is an enlarged partial view of the outlined section shown in FIG. 3A;

FIG. 4 is a block diagram of a treatment system in accordance with one or more aspects of the invention;

FIG. 5 shows a potential use of a treatment system in accordance with one or more aspects of the invention;

FIG. 6 is a first series of images showing the impact of microparticles on a sample in accordance with one or more aspects of the invention;

FIG. 7 is a second series of images showing a microparticle's impact on a sample in accordance with one or more aspects of the invention; and

FIG. 8 is a functional block diagram illustrating one example of a method for forming a treatment device in accordance with one or more aspects of the present invention.

### DETAILED DESCRIPTION

Several pharmacological therapies have been introduced to treat solid tumors, including chemotherapy and radiotherapy. Localized treatment on aggressive solid tumors can be clinically challenging. In cases where local high bioavailability is required to treat difficult-to-access tumors, localized delivery platforms are preferable to traditional delivery methods.

Localized and targeted delivery reduces risks for patients that stem from high drug level toxicity in the body that is caused by systemic delivery. Localized treatment also overcomes the difficulty for certain drugs to bypass anatomical barriers, such as the blood-brain barrier (BBB), that limit uptake of pharmacological drugs. A number of radio-therapies already exist to treat tumors, including use of conventional external beam radiation therapy (2DXRT) or radioisotope therapy (RIT) for targeted therapy. While successful in treating tumors, side effects of these treatments, including radiation of healthy tissue, combined with expensive treatment costs pose a challenge to the medical care system. There is therefore a clinical need for treatment of solid tumors that are difficult to access, especially tumors that may be deemed inoperable that is capable of providing localized treatment using a minimally invasive technique that is cost-effective.

The treatment systems and methods disclosed herein offer a therapeutic modality for treatment of target conditions such as solid tumors that may be characterized as surgically inaccessible or difficult to access. The treatments disclosed herein may give localized pharmacological or radiological treatment that minimizes toxicity and harm to healthy tissues. The technique relies on laser-induced acceleration of microparticles to high speeds for local, targeted delivery of therapeutic agents such as active pharmaceutical ingredients (APIs) to a target condition, such as inoperable tumors. These systems and processes provide an accurate method to spatially control dispersion and penetration of solid tumors.

Although the above discussion is directed to a specific application of tumors, the methods and systems discussed herein may be used to treat a target condition, which in some instances is a tumor, but may also refer to any one of a number of different physiological conditions that may benefit from receiving localized concentrations of one or more therapeutic agents.

According to at least one embodiment, monodisperse microparticles comprising therapeutic agents may be deposited on a substrate coated with a laser-absorbing material. This substrate functions as a launch platform that may be coupled with a source of light energy to accelerate the microparticles to a velocity that launches them from the substrate and projects them into a target condition, such as a tumor. A wide variety of therapeutic agents may be delivered to the target condition using the microparticles, including radioactive and pharmaceutical materials. The launch platform is interchangeable and may also be replaced after use. In some instances, the launch platform is disposable. As discussed further below, the launch platform may be connected to a housing of a treatment device. Launch platforms may be configured with different therapeutic agents for purposes of targeting specific conditions and/or applications.

The aspects disclosed herein in accordance with the present invention, are not limited in their application to the details of construction and the arrangement of components set forth in the following description or illustrated in the accompanying drawings. These aspects are capable of assuming other embodiments and of being practiced or of being carried out in various ways. Examples of specific implementations are provided herein for illustrative purposes only and are not intended to be limiting. In particular, acts, components, elements, and features discussed in connection with any one or more embodiments are not intended to be excluded from a similar role in any other embodiments.

Also, the phraseology and terminology used herein is for the purpose of description and should not be regarded as limiting. Any references to examples, embodiments, components, elements or acts of the systems and methods herein referred to in the singular may also embrace embodiments including a plurality, and any references in plural to any embodiment, component, element or act herein may also embrace embodiments including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. In addition, in the event of inconsistent usages of terms between this document and documents incorporated herein by reference, the term usage in the incorporated reference is supplementary to that of this document; for irreconcilable inconsistencies, the term usage in this document controls.

In accordance with various aspects, a system for treating a target condition is provided. As discussed above, the target condition may be a tumor, including inoperable tumors, although other medical conditions are within the scope of this disclosure. For example, the target condition may be an infection, lesion, inflammation, cell and tissue damage, or benign tissue growth. In some embodiments, the target condition may be a dermatological disease, including cancer, such as carcinoma, and immune diseases, such as rheumatoid arthritis. The target condition may be any medical or physiological condition that may benefit from receiving localized concentrations of one or more therapeutic agents.

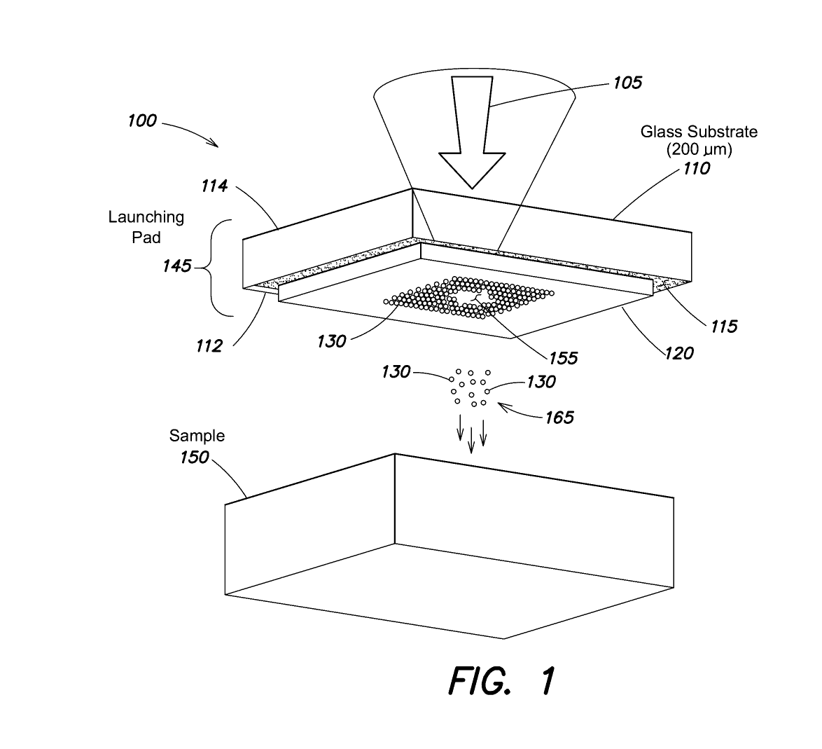

Referring to FIG. 1, one embodiment of a treatment system, shown generally at 100, comprises a launch platform 145, otherwise referred to herein as a “launch pad,” and a source of light 105. As explained in further detail below, one surface of the launch platform 145 includes microparticles 130 comprising therapeutic agents that can be projected at high speeds into the target condition 150 using light source 105 directed the opposite surface (shown as surface 114 in FIG. 1) of the launch platform 145.

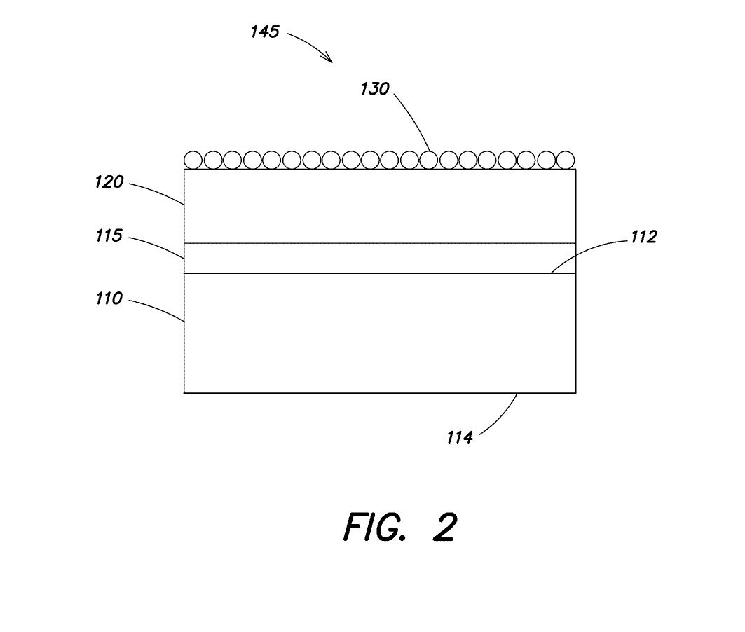

According to one embodiment, the launch platform 145 comprises a substrate 110, a layer of light absorption material 120, and a layer of microparticles 130. The microparticles 130 comprise at least one therapeutic agent, as discussed in further detail below. In some embodiments, the launch platform 145 may also include a conductive layer, such as a layer of metal 115. A cross-sectional view of a launch platform 145 is shown in FIG. 2.

The substrate 110 may be constructed from one or more materials that are transparent to one or more wavelengths of light. The substrate 110 may be transparent to visible light, infrared light, and/or ultraviolet light provided by the light source 105. The substrate 110 may be made of a material such as glass or a polymer configured to be transparent to a wavelength of light of interest. In addition, the substrate 110 material may be chosen such that it does not react or otherwise interact or interfere with the physical and chemical processes discussed herein. According to some embodiments, the substrate 110 is transparent to one or more wavelengths of infrared light. The substrate 110 functions to transmit the light energy emitted from the light source 105 to one or more layers deposited on the surface of the substrate 110. Therefore, the substrate 110 may be made from material that does not absorb a sufficient amount of the emitted light energy, and in some instances may transmit nearly all of the emitted light energy to one or more layers deposited on a surface of the substrate 110.

The substrate 110 may be a thickness that provides adequate mechanical strength to withstand physical and chemical processes discussed herein while not impeding these processes. In some embodiments, the substrate 110 is sized and shaped to fit within the housing of a treatment device, as discussed in further detail below. According to one embodiment, the substrate 110 is between about 100 microns to about 1000 microns in thickness. In one embodiment, the substrate 110 is about 200 microns thick, although thicker and thinner substrates are within the scope of this disclosure.

In some embodiments, a layer of light absorption material 120, also referred to herein as simply “absorption material” or as a “photoconductive material,” may be deposited on the substrate 110 of the launch platform 145. The layer of light absorption material 120 may function to absorb at least a portion of the light energy emitted from the light source 105 through substrate 110. The layer of light absorption material 120 may be selected or otherwise configured to absorb one or more desired wavelengths of light, such as visible light, infrared light, and/or ultraviolet light. In one embodiment, the absorption material 120 absorbs one or more wavelengths of infrared light. For instance, the absorption material 120 may absorb one or more wavelengths in the near infrared (NIR) region of the electromagnetic spectrum (750-1400 nm).

According to certain embodiments, the light energy absorbed by the absorption material 120 may be in the form of thermal energy, such as heat. For example, the absorption material 120 may become heated which causes the absorption material 120 to vaporize or otherwise expand to a degree such that microparticles 130 deposited on the surface of the absorption material 120 accelerate to a velocity such that they are launched from the surface of the absorption material 120. According to some embodiments, the light energy absorbed by the absorption material 120 may cause the absorption material 120 to chemically react such that it accelerates the microparticles 130 to a velocity sufficient for the microparticles 130 to be launched from the surface of the absorption material 120. In some instances, the reaction is a vaporization reaction that releases one or more gases that cause the microparticles 130 to accelerate.

According to some embodiments, the light absorption material 120 may be a polymer. The polymer may be chosen for its ability to absorb light energy emitted from the light source 105 at a desired wavelength, and for its ability to interact with the light energy such that one or more microparticles 130 deposited on the surface of the absorption material 120 can be launched at a velocity sufficient to penetrate a target sample, such as a tumor. In one embodiment, the light absorption material 120 is polydimethylsiloxane (PDMS). In some embodiments, the light absorption material 120 may include one or more additives, such as a dye or other material that functions to enhance the light energy absorption properties of the material. In accordance with one or more embodiments, the light absorption material 120 may also function to enhance the formation of a monolayer of microparticles 130. For instance, the absorption material 120 may have a certain molecular structure or pattern that allows for microparticles 130 to be arranged in a monolayer.

The layer of light absorption material 120 may be of any thickness suitable for the processes discussed herein. The layer of light absorption material 120 may be thick enough to absorb light energy emitted from the light source 105 such that at least a portion of a plurality of microparticles 130 deposited on the surface of the absorption material 120 are capable of being launched at a speed sufficient to penetrate a target condition, such as a tumor. In some embodiments, the layer of absorption material 120 is several microns thick. In one embodiment, the layer of absorption material 120 may be between about 1 micron and 100 microns thick. In some embodiments, the layer of absorption material is about 10 microns thick.

According to some embodiments, the launch platform 145 further comprises a layer of conductive material 115. For instance, the launch platform may comprise a layer of metal material 115, although other conductive materials are also within the scope of this disclosure. As shown in FIGS. 1 and 2, the layer of metal 115 may be deposited on a surface of the substrate 110. The layer of absorption material 120 may be formed on the layer of metal 115. The metal 115 may be any one or more metal materials, including metal alloys, homogeneous elements, metal-containing materials and mixtures thereof. According to one embodiment, the metal 115 may be a highly conductive material, such as gold. The thickness of the metal 115 may depend on the application and aspects of the device, such as the source of light energy 105 as well as the light absorption material 120. In some embodiments, the metal 115 is deposited in a thin layer. For example, the metal 115 may be between about 10 nm and about 100 nm. In one embodiment, the layer of metal 115 is about 50 nm thick.

The layer of metal 115 may function to enhance one or more properties of the launch platform 145 and the materials disposed thereon. For instance, the layer of metal 115 may enhance the ability of the microparticles 130 to form or otherwise be arranged into a self-assembled monolayer. For instance, the metal material 115 may have a certain molecular structure or pattern that allows for microparticles 130 to be arranged in a monolayer on the surface of the absorption layer 120. The layer of metal 115 may also function to conduct or transmit light energy emitted from the light source 105 and may therefore aid in accelerating the microparticles 130 from the absorption material 120. At least a portion of the energy emitted from the light source 105 may pass into the metal 115 and this energy may be transferred to the absorption layer 120. For example, the light energy may heat the layer of metal 115 and this thermal energy may be transferred to the absorption layer 120. In some embodiments, the metal 115 may be chemically inert to the reaction processes discussed herein and may therefore be chosen for this ability to not detrimentally interfere with other mechanisms occurring during operation of the treatment system or device.

According to certain embodiments, the launch platform 145 may further comprise a layer of microparticles 130. The layer of microparticles 130 may be deposited on the layer of absorption material 120 and may form an outer surface of the launch platform 145. In some embodiments, the layer of microparticles 130 are arranged as a self-assembled monolayer, as shown in FIGS. 1 and 2. As such, the microparticles 130 may be deposited such that they are one microparticle in thickness. In some embodiments, the microparticles 130 may also be closely packed. This type of configuration may allow for more targeted control of the microparticles 130 as they are directed to the target condition 150. The microparticles 130 may have an approximate diameter or characteristic linear dimension in a range of about 1 and about 10 microns. Larger and smaller sized microparticles are also within the scope of this disclosure. The size of the microparticle 130 may depend on the therapeutic agent, as discussed below, as well as the type of application. Microparticles 130 of different dimensions may also be deposited as a monolayer onto the launch platform 145.

The microparticles 130 may comprise at least one therapeutic agent. Therapeutic agents include any molecule that can be associated with the microparticle and used in the systems and methods of the present invention. They can be purified molecules, substantially purified molecules, molecules that are one or more components of a mixture of compounds, or a mixture of a compound with any other material. The molecules can be organic or inorganic chemicals, radioisotopes, pharmaceutical compounds, pharmaceutical salts, pro-drugs, or biomolecules, and all fragments, analogs, homologs, conjugates, and derivatives thereof. Biomolecules include, without limitation, proteins, polypeptides, nucleic acids, lipids, polysaccharides, monosaccharides, and all fragments, analogs, homologs, conjugates, and derivatives thereof. Agents can also be an isolated product of unknown structure, a mixture of several known products, or an undefined composition comprising one or more compounds. Examples of undefined compositions include cell and tissue extracts, growth medium in which prokaryotic, eukaryotic, and archaebacterial cells have been cultured, fermentation broths, protein expression libraries, and the like. Therapeutic agents can be provided in or otherwise associated with a carrier such as a pharmaceutically acceptable carrier.

The terms “therapeutic agents,” “biologically active agents,” “drugs,” “pharmaceutically active agents,” “pharmaceutically active materials,” and other related terms may be used interchangeably herein and include genetic therapeutic agents, non-genetic therapeutic agents and cells. Numerous therapeutic agents can be employed in conjunction with the present disclosure, including those used for the treatment of a wide variety of diseases and conditions besides tumor treatment (i.e., the prevention of a disease or condition, the reduction or elimination of symptoms associated with a disease or condition, or the substantial or complete elimination of a disease or condition). Numerous therapeutic agents are described herein.

Other non-limiting examples of therapeutic agents include antioxidants, anti-angiogenic agents, calcium entry blockers (e.g., verapamil, diltiazem, nifedipine), steroidal and nonsteroidal anti-inflammatory agents (e.g., dexamethasone, prednisolone, corticosterone, budesonide, estrogen, acetyl salicylic acid, sulfasalazine, mesalamine, etc.), anesthetic agents (e.g., lidocaine. bupivacaine and ropivacaine), protein kinase and tyrosine kinase inhibitors, anti-proliferative agents, cytostatic agents (i.e., agents that prevent or delay cell division in proliferating cells, for example, by inhibiting replication of DNA or by inhibiting spindle fiber formation) (e.g., toxins, methotrexate, adriamycin, radionuclides, protein kinase inhibitors such as staurosporin and diindoloalkaloids, etc.), agents that inhibit intracellular increase in cell volume (i.e., the tissue volume occupied by a cell) such as cytoskeletal inhibitors (e.g., colchicine, vinblastin, cytochalasins, paclitaxel, etc.) or metabolic inhibitors e.g., staurosporin, Pseudomonas exotoxin, modified diphtheria and ricin toxins, etc.), trichothecenes (e.g., a verrucarin or roridins), agents acting as an inhibitor that blocks cellular protein synthesis and/or secretion or organization of extracellular matrix (i.e., an “anti-matrix agent” such as colchicine or tamoxifen), various pharmaceutically acceptable salts and derivatives of the foregoing, and combinations of the foregoing, among other agents.

Examples of therapeutic agents which may be used in the compositions of the present disclosure thus include toxins (e.g., ricin toxin, radioisotopes, or any other agents able to kill undesirable cells, such as those making up cancers and other tumors such as neuroendocrine tumors) and agents that arrest growth of undesirable cells.

In accordance with some embodiments, the therapeutic agent may be an anti-tumor agent. Non-limiting examples of anti-tumor agents include radioisotopes such as ⁹⁰Y, ³²P, ¹⁸F, ¹⁴⁰La, ¹⁵³Sm, ¹⁶⁵Dy, ¹⁶⁶Ho, ¹⁶⁹Er, ¹⁶⁹Yb, ¹⁷⁷Lu, ¹⁸⁶Re, ¹⁸⁸Re, ¹⁰³Pd, ¹⁹⁸Au, ¹⁹²Ir, ⁹⁰Sr, ^{m}In or ⁶⁷Ga, antineoplastic/antiproliferative/anti-miotic agents including antimetabolites such as folic acid analogs/antagonists (e.g., methotrexate, etc.), purine analogs (e.g., 6-mercaptopurine, thioguanine, cladribine, which is a chlorinated purine nucleoside analog, etc.) and pyrimidine analogs (e.g., cytarabine, fluorouracil, etc.), alkaloids including taxanes (e.g., paclitaxel, docetaxel, etc.), alkylating agents such as alkyl sulfonates, nitrogen mustards (e.g., cyclophosphamide, ifosfamide, etc.), nitrosoureas, ethylenimines and methylmelamines, other aklyating agents (e.g., dacarbazine, etc.), antibiotics and analogs (e.g., daunorubicin, doxorubicin, idarubicin, mitomycin, bleomycins, plicamycin, etc.), platinum complexes (e.g., cisplatin, carboplatin, etc.), antineoplastic enzymes (e.g., asparaginase, etc.), agents affecting microtubule dynamics (e.g., vinblastine, vincristine, colchicine. Epo D, epothilone), caspase activators, proteasome inhibitors, angiogenesis inhibitors (e.g., statins such as endostatin, cerivastatin and angiostatin, squalamine, etc.), rapamycin (sirolimus) and its analogs (e.g., everolimus, tacrolimus, zotarolimus, etc.), etoposides, and many others (e.g., hydroxyurea, flavopiridol, procarbizine, mitoxantrone, campothecin, etc.), various pharmaceutically acceptable salts and derivatives (e.g., esters, etc.) of the foregoing, and combinations of the foregoing, among other agents. Further therapeutic agents include thrombogenic agents such as homocysteine.

Other non-limiting examples of therapeutic agents include chemical ablation agents (materials whose inclusion in the formulations of the present disclosure in effective amounts results in necrosis or shrinkage of nearby tissue upon impact) including osmotic-stress-generating agents (e.g., salts, etc.). Specific examples of chemical ablation agents from which suitable agents can be selected include the following: basic agents (e.g., sodium hydroxide, potassium hydroxide, etc.), acidic agents (e.g., acetic acid, formic acid, etc.), enzymes (e.g., collagenase, hyaluronidase, pronase, papain, etc.), free-radical generating agents (e.g., hydrogen peroxide, potassium peroxide, etc.), other oxidizing agents (e.g., sodium hypochlorite, etc.), tissue fixing agents (e.g., formaldehyde, acetaldehyde, glutaraldehyde, etc.), coagulants (e.g., gengpin, etc.), non-steroidal anti-inflammatory drugs, contraceptives (e.g., desogestrel, ethinyl estradiol, ethynodiol, ethynodiol diacetate, gestodene, lynestrenol, levonorgestrel, mestranol, medroxyprogesterone, norethindrone, norethynodrel, norgestimate, norgestrel, etc.), GnRH agonists (e.g. buserelin, cetorelix, decapeptyl, deslorelin, dioxalan derivatives, eulexin, ganirelix, gonadorelin hydrochloride, goserelin, goserelin acetate, histrelin, histrelin acetate, leuprolide, leuprolide acetate, leuprorelin, lutrelin, nafarelin, meterelin, triptorelin, etc.), antiprogestogens (e.g., mifepristone, etc.), selective progesterone receptor modulators (SPRMs) (e.g., asoprisnil, etc.), various pharmaceutically acceptable salts and derivatives of the foregoing, and combinations of the foregoing, among other agents.

According to some embodiments, the microparticles 130 may comprise one or more imaging agents in amounts useful to enhance in vivo imaging of the particles. The imaging agent may be provided in microparticle cores, particle shells, or both. Non-limiting examples of imaging agents include (a) contrast agents for use in conjunction with magnetic resonance imaging (MRI), including contrast agents that contain elements with relatively large magnetic moment such as Gd(III), Dy(III), Mn(II), Fe(III) and compounds (including chelates) containing the same, such as gadolinium ion chelated with diethylenetriaminepentaacetic acid, and (b) contrast agents for use in connection with x-ray fluoroscopy, including metals, metal salts and oxides (particularly bismuth salts and oxides), and iodinated compounds, among others.

In certain embodiments, the microparticles 130 may be rendered magnetic (e.g., they contain magnetized materials) or are rendered susceptible to magnetic fields (e.g., they contain paramagnetic or ferromagnetic materials such as iron). For example, magnetic, paramagnetic or ferromagnetic materials may be provided in microparticle cores, particle shells, or both. Non-limiting examples of magnetic, paramagnetic or ferromagnetic materials include metals, alloys or compounds (e.g., oxides, etc.) of certain transition, rare earth and actinide elements, and iron or iron oxide.

Microparticles that include radioisotopes such as lutetium-177 may be used for targeted RIT of unresectable tumors and magnetic iron oxide microparticles may be used in systemic radiation therapy as a drug vehicle and also as a contrast imaging agent for MRI. Likewise, biodegradable microparticles, such as PLGA particles, may be used as drug carriers for controlled drug delivery. The techniques discussed herein may be used to successfully and reliably accelerate to high speeds lutetium microparticles, as well as PLGA microparticles embedded with iron oxide magnetic nanoparticles and dexamethasone.

The microparticles 130 may be prepared using any suitable technique. Techniques for forming microparticles in accordance with the disclosure include those wherein microparticles are formed from one or more liquid phases (e.g., solutions, suspensions, polymer melts) that contain the therapeutic agent of interest. The microparticles 130 may also be formed in a liquid phase that includes further ingredients such as solvents, other therapeutic agents, imaging agents, magnetic/paramagnetic/ferromagnetic materials, etc. In some embodiments, microparticles are formed which have a core-shell structure, whereas in other embodiments, the microparticles are formed of a core material without the shell. Either the shell or the core may contain one or more therapeutic agents and/or other materials, such as a metal, an imaging agent, a polymer, or a drug carrier. For example, the microparticles 130 may be metal microparticles such a tungsten or radioisotopes as lutetium, or a polymer microparticles such as a resin material. In some embodiments, the microparticle comprises a biodegradable material, such as PGLA (poly(lactic-co-glycolic acid) particles that further comprise an imaging agent such as a fluorescent dye or iron oxide. In some embodiments, the microparticles are a pharmaceutical, such as a drug, that is coated with a substance that controls the drug's release upon impact into the target condition.

In some embodiments, microparticles 130 comprising different therapeutic agents may be disposed on the launch platform. For instance, each microparticle 130 may comprise two or more different therapeutic agents, or two or more microparticles 130 comprising different therapeutic agents may be used. Thus, multiple therapeutic agents may be delivered to the tumor site.

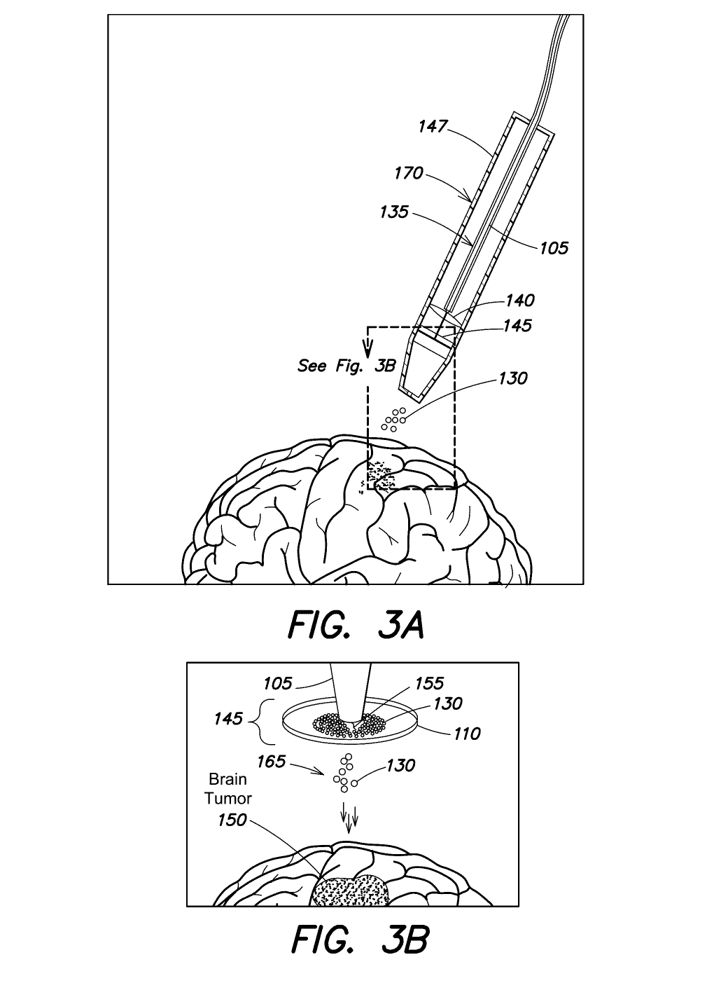

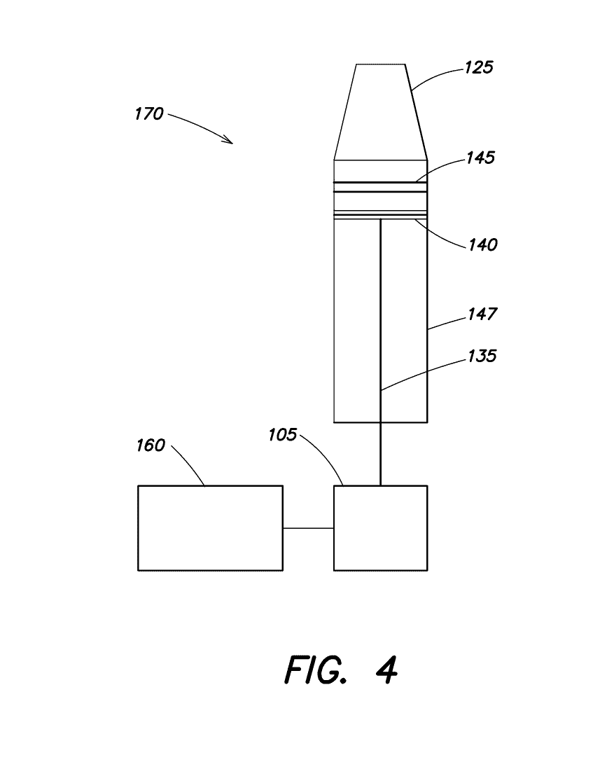

According to at least one embodiment, a treatment device is provided that is configured to deliver microparticles to a target condition such as a tumor. Referring to FIGS. 3A and 4, one example of such a treatment device 170, otherwise referred to herein as a “treatment probe” is shown. The treatment device 170 comprises a housing 147, a launch platform 145 positioned within the housing 147, and a light source 105.

The housing 147 may be made from a rigid material to withstand physical and chemical processes discussed herein. In some embodiments, the housing 147 may be made from a material that is capable of being sanitized or otherwise disinfected, such as a metal or metal alloy or one or more polymer materials. Disinfection of the housing 147 may become necessary in instances where the housing 147 is placed in proximity to tissue or other substances where there is a risk of infection.

The launch platform 145 of the treatment device 170 may be provided and characterized as previously discussed. For instance, the launch platform 145 may comprise a substrate 110, a layer of metal 115 deposited on a first surface 112 (see FIGS. 1 and 2) of the substrate 110, a layer of light absorption material 120 deposited on the layer of metal 115, and a layer of microparticles 130 comprising at least one therapeutic agent deposited on the layer of absorption material 120.

According to various embodiments, the launch platform 145 of the treatment device 170 is interchangeable. As used herein, the term “interchangeable” refers to removable, replaceable, and/or interchangeable. For example, the launch platform 145 may be connected to the housing 147 such that it can be removed from the housing and replaced with a different launch platform 145. For instance, the housing 147 may have an opening where launch platforms may be positioned and interchanged. The housing 147 may include a removable portion that allows access to the launch platform. Launch platforms may be configured with one or more different microparticles 130 that contain different therapeutic agents or other materials, such as image agents or drug carriers. A launch platform 145 may be removed once at least a portion of the microparticles 130 have been delivered to a target condition 150, shown as a brain tumor in FIGS. 3A and 3B. The housing 147 may therefore be configured with an opening mechanism such that the launch platform 145 may be removed and replaced. For instance, the treatment device may be configured with a removable tip 125 that is clipped or threaded onto the main body of the housing 147. The removable tip 125 may also be conical in shape so as to minimize or otherwise reduce scattering of the plume 165 of microparticles 130 emitted from the device. The conical shape may also assist in focusing the light energy emitted from the light source 105.

The treatment device 170 also comprises, or is connectable to, a light source 105. According to some embodiments, the light source 105 may be configured as a laser light source that is coupled to an optical fiber 135, as shown in FIGS. 3A and 4. The optical fiber 135 may be configured to transmit light energy emitted form the light source 105. For example, a first end of the optical fiber may be coupled or otherwise in optical communication with the laser light source, and a second end of the optical fiber 135 may terminate in proximity to a focusing lens 140 (discussed further below). Light energy emitted from the light source 105 may therefore be transmitted or otherwise conducted through the optical fiber 135 so that it may be delivered to the launch platform 145.

In some embodiments, the treatment device 170 further comprises a focusing lens 140, as shown in FIGS. 3A and 4, disposed between the light source 105 and a second surface 114 (see FIGS. 1 and 2) of the substrate 110 of the launch platform 145. The focusing lens 140 may be configured to focus light energy passing therethrough. The focusing lens 140 may focus the light energy onto at least a portion of the second surface 114 of the substrate 110. In accordance with one embodiment, the focusing lens 140 may be configured as one or more miniaturized optics that are arranged to focus the laser light onto the launch platform 145.

The light source 105 may be of any wavelength that is suitable for a desired application. In some embodiments, the laser is configured to emit an infrared wavelength, including NIR wavelength(s). Furthermore, one or more properties of the light energy emitted from the light source 105 may be controlled by a controller 160, as shown in FIG. 4. For instance, in embodiments where the light source 105 is a laser light source, such as a pump laser, the controller 160 may be configured to control at least one of the laser pulse energy and pulse duration. The controller 160 may also be configured to control power to the light source 105. In some embodiments, the controller 160 may also be configured to control the wavelength of light emitted from the light source 105. According to one embodiment, the laser light source may be a pump laser including a laser diode that can use continuous wave (CW) or quasi-CW light, which may comprise pulses broader than approximately 100 picoseconds. For example, according to one embodiment, the laser may be an 800 nm quasi-CW laser that is pulsed at 300 ps and is applied at a pulse energy of 1 mJ. In some embodiments, the light source may be a tunable light source that may be operated in continuous wave mode or in a pulsed mode. A tunable light source may be modulated by the controller 160. For example, the controller 160 may tune or otherwise control or modulate the primary frequency ωₚᵣ. The controller 160 may also modulate the light source 105 to produce pulses of a certain format.

According to some embodiments, a method for treating a target condition is provided. The method may comprise locating a target condition in a subject, positioning the treatment device to be in proximity to the target condition, applying a source of light energy to a launch platform to accelerate a plurality of microparticles comprising at least one therapeutic agent to a predetermined velocity, and directing the plurality of microparticles at the targeted condition. The predetermined velocity may be a velocity that causes the plurality of microparticles to penetrate at least a portion of the target condition. In accordance with some embodiments, a treatment system is provided that is configured to perform one or more functions of the treatment method.

During operation of the treatment device 170, light energy emitted from the light source 105 is transmitted through the optical fiber 135 and focused by the focusing lens 140 onto at least a portion of the second surface 114 of the substrate 110 of the launch platform 145, as shown in FIG. 1. In some embodiments, the first surface 112 and the second surface 114 of the substrate 110 are positioned opposite from one another. However, other configurations are also within the scope of this disclosure. For instance, one or more portions of the substrate 110 may be coated or otherwise contain a reflective material that allows for the first surface 112 to be disposed at an angle to the second surface 114.

As previously described, light energy emitted by the light source 105 is transmitted through the substrate 110 and transferred to the absorption material 120. One or more reaction mechanisms caused by chemical and/or thermal processes caused by the light energy accelerate at least a portion of the microparticles 130 disposed on the surface of the absorption layer 120 to a velocity sufficient to penetrate at least a portion of the target condition. For instance, the light source 105 may be a laser light source such that upon intense laser excitation and absorption by the absorption material 120, vaporization and the rapid expansion of gas causes acceleration of the microparticles to speeds sufficient to penetrate a target condition such as a target condition contained within human tissue. Depending on the microparticle used and one or more properties of the laser light source, including the pulse energy and duration, the microparticles may reach velocities ranging from about 0.4 km/s to about 4 km/s.

The processes discussed herein may be described as a laser ablation technique. Laser ablation generally refers to the process of removing material from a solid and is based on the absorption of laser photons by the absorption material. The ablation mechanism is a complex combination of photochemical and photothermal reactions that is specific for each laser and absorption material and is dependent on the laser's characteristics and the properties of the absorption material. The ablation mechanism is best shown in FIGS. 1 and 3B. The ablated area 155 indicates where microparticles 130 have accelerated and launched from the surface of the absorption material 120 to travel to the target condition 150. In some embodiments, the light source 105 may output a sequence of laser pulses. The beam size at the surface of the substrate, pulse energy, and pulse duration of the laser light source may be selected so that each pulse from the laser light source that irradiates an area of the substrate removes by ablation the microparticles 130 from at least a portion of the irradiated area, in some embodiments, the ablated area 155 may have a diameter or characteristic linear dimension in a range of about 10 microns to about 100 microns.

The rate of acceleration of the microparticle 130 may depend on one or more factors. For instance, the laser wavelength, pulse energy, and pulse duration may affect the acceleration of the microparticles 130. For instance, shorter laser pulses may enable more localization of energy delivery to the absorption material in smaller dimensions than longer laser pulses. In addition, the spatial configuration of the launched microparticles, such as the width of the launched plume 165 (see FIGS. 1 and 3B) of microparticles 130, may be dependent on one or more factors, such as the beam size, which may be a function of the width of the optic fiber 135 and/or the configuration of the focusing lens 140. For instance, smaller or larger widths of the plume 165 of microparticles 130 may be controlled by varying the dimensions of the focusing lens 140 and/or the pulse energy and duration of the laser. This control aids in allowing the device to provide location-specific and targeted application of the microparticles 130.

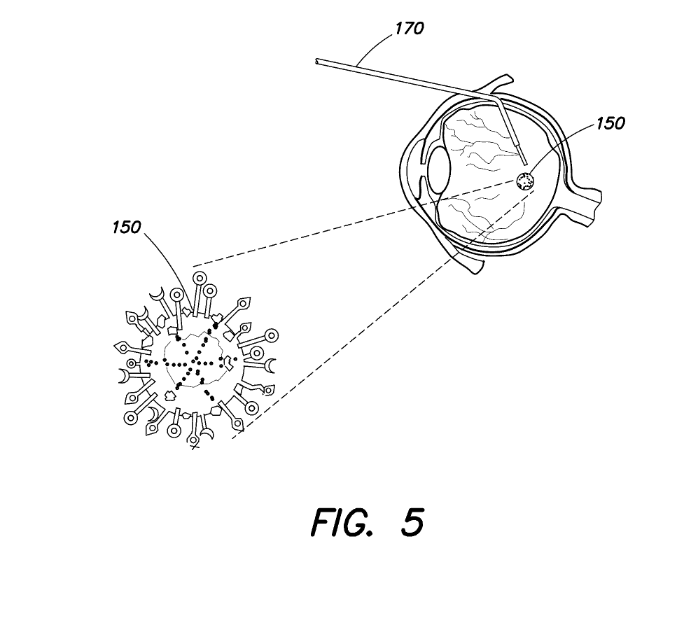

FIG. 5 illustrates a specific application of a treatment device 170 for a target condition 150 that includes an eye cancer, such as retinoblastoma, which forms in the rear portion of the eye and may therefore be difficult to reach or inoperable using standard surgical methods. As shown, the treatment device 170 may be used to deliver therapeutic agents to the site of the retinoblastoma. In some embodiments, the treatment device 170 may be sized or otherwise configured to penetrate a portion of the eye for delivering the microparticles to the target condition 150, and in other embodiments, the treatment device 170 may be placed in proximity to the eye such that microparticles launched from the treatment device are able to penetrate the eye to a degree such that they are delivered to the target condition 150.

The treatment device and method discussed herein may also be adapted for other surgical equipment such as endoscopes. The invention may be used to treat unresectable tumors, such as gliomas or GBT, with minimal intrusion due to the ability of the device and method to provide laser-assisted delivery of therapeutic agents.

In accordance with some embodiments, the treatment device 170 is configured to determine or otherwise locate a target condition 150 in a subject. For instance, in some applications, it may be difficult to determine exactly where a tumor or other target condition is positioned within a larger tissue sample. For instance, the tumor may be embedded deep within the tissue or at a location that is hidden by other objects, such as bone tissue. The treatment device 170 may be equipped with a location mechanism, including an imaging component, such as a fiber optic, and/or camera, or an ultrasound device, or a sensor configured to detect a substance present in the tumor or target condition. One or more of these techniques may make it easier to determine how far from the emitting end of the treatment device 170 the target condition is located. This information may then be fed back to the controller 160, which may react by adjusting or setting physical parameters of the light energy from the light source 105, such as the wavelength, pulse energy, and/or pulse duration. In some embodiments, this information may also dictate or otherwise influence the choice of a launch platform, and may also influence the choice of the treatment device itself. For instance, the beam size and resulting plume emitted from the launch platform may be influenced by the size of the housing of the treatment device 170 and/or the configuration of the focusing lens positioned within the housing.

According to another embodiment, the treatment device 170 may be positioned in proximity to a target condition. For instance, the location information described above may be used to position the treatment device a certain distance from the target condition. This allows for a carefully controlled dispersion and penetration of the target condition by the plume of microparticles 130 emitted from the treatment device 170.

### Example

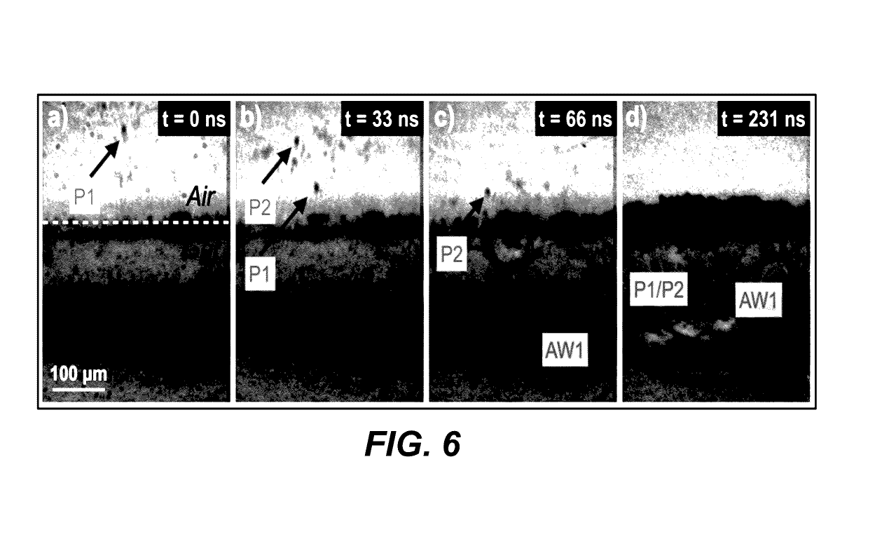

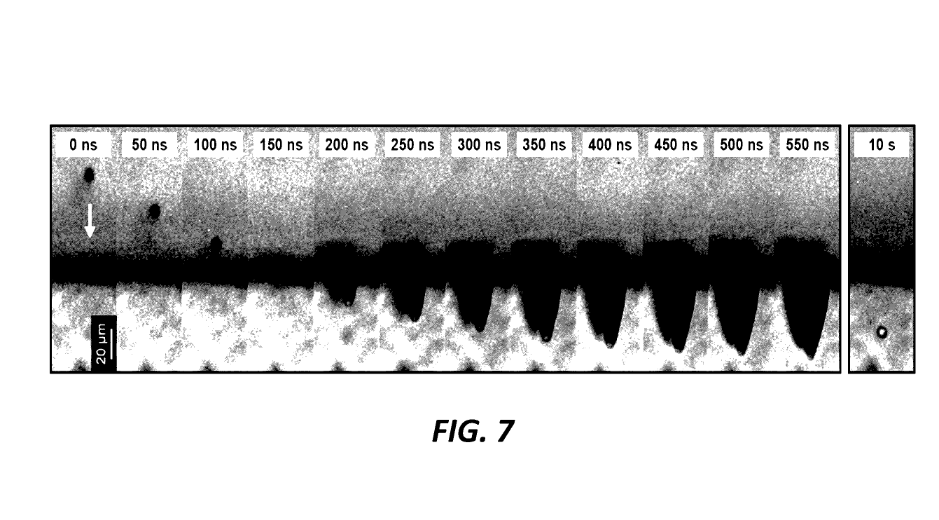

A test example showing a microparticle's impact on a simulated tissue sample is illustrated in FIGS. 6 and 7. For this test, a quasi-cw 800 nm laser light source and a high speed camera were used to record the side view images shown in FIGS. 6 and 7. FIG. 6 shows a series of images taken at different times (0 ns, 33 ns, 66 ns, and 231 ns) capturing the impact of silica beads having a diameter of 7.4 microns on PDMS-based gel whose fabrication was tailored to mimic the mechanical response of a solid tumor. By varying the laser pulse energy and choice of microparticle, the microparticle's impact speed and resulting depth of penetration can be controlled. The images of FIG. 6 show how microparticles labeled as P1 and P2 are launched from a launch platform and arrive from a top side of the field of view at times t=0 ns and t=33 ns, and impact and penetrate the sample with a speed of about 3 km/s. Spherical acoustic waves (labeled as “AW1”) are generated upon impact and can be followed for hundreds of nanoseconds inside the material, as shown at times t=66 ns and t=231 ns. The air-gel interface is represented by the white dashed line at t=0 ns. The series of photographs allows for the trajectory and path of the microparticles P1 and P2 to be followed before impact and as P1 penetrates inside the gel to give rise to an acoustic wave AW1. FIG. 7 is a similar series of time-separated images taken of a high speed particle as it impacts a transparent gel sample (PDMS-based gel configured to emulate a solid tumor). The images are shown in 50 ns increments and show the particle as it moved downward through the air and into the gel. The particle penetrates the gel sample to a depth of 20 microns after about 200 ns and at 10 seconds is shown to have completely penetrated the gel to a depth of about 40 microns. Both sets of time-sequence photographs shown in FIGS. 6 and 7 indicate that all particles penetrated the gel.

A prophetic example of a treatment device includes a laser light source having a wavelength of 800 nm and is configured as a quasi-CW laser that is pulsed at 300 ps and applies a pulse energy of 1 mJ at a surface of a launch platform. The launch platform includes a glass substrate having a thickness of 200 microns, and a 50 mm thick layer of gold metal is deposited on a first surface of the glass substrate. A 10 micron thick layer of PDMS is deposited on the gold film. Microparticles having a size in a range of from between 1 and 10 microns are deposited on a surface of the PDMS layer. The light energy emitted from the laser is sufficient to launch the microparticles from the PDMS layer at a velocity ranging from 0.4-4 km/s, which is a speed sufficient for the microparticles to penetrate soft human tissues.

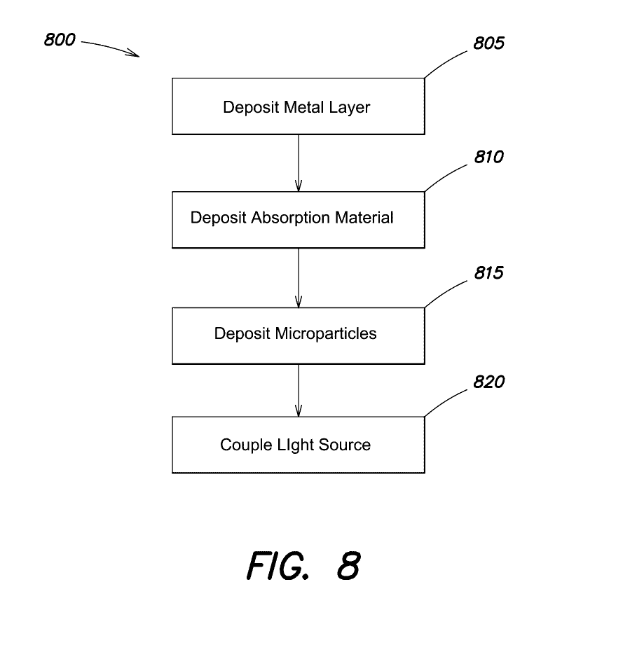

In accordance with some embodiments, a method for forming a treatment device is provided. A functional block diagram for forming one embodiment of a treatment device is shown in FIG. 8. According to this embodiment, the FIG. 8 outlines a method 800 suitable for forming a device such as that shown in FIGS. 1, 3A, and 4.

The process 800 may include depositing a layer of metal onto a first surface of a substrate at act 805. The metal and the substrate may be characterized as described above. For instance, the substrate may be a glass substrate that is transparent to one or more wavelengths of electromagnetic radiation, such as IR or NIR wavelengths of light. In some embodiments, the metal may function to conduct thermal energy from the light energy to the layer of absorption material, as described above. The metal may be deposited using any one or more metal deposition techniques, including sputtering or evaporation methods, such as physical vapor deposition (PVD).

A layer of light absorption material may be deposited onto the layer of metal at act 810. The light absorption material may be a material as described above. The light absorption material may be deposited using a spin-coat or spray-coat method or a vapor deposition method, such as vapor deposition polymerization in instances where the absorption material is a polymer.

At act 815, a layer of microparticles is deposited onto the layer of absorption material. The microparticles may comprise at least one therapeutic agent as discussed above. The microparticles may be deposited such that they form a self-assembled monolayer. Therefore, the microparticles may be deposited as a film that is one microparticle in thickness and in some instances may also be closely packed. The microparticles may be deposited using any one of a number of different techniques, such as electrostatic deposition, e.g., electrostatic spray, printing, e.g., ink jet techniques, and evaporation techniques, e.g., CVD or PVD. The selected deposition depends on the type of microparticle being deposited. For instance, radioisotopes or metals may be deposited using sputtering or PVD methods.

The method may also comprise coupling a light source to a second surface of the substrate at act 820. For instance, a laser light source may be coupled to or otherwise placed in proximity to the second surface of the substrate such that light energy is transmitted to the substrate surface. The method may also comprise positioning a focusing lens in between the light source and the second surface of the substrate. As discussed above, the focusing lens may comprise miniaturized optics that function to focus the light energy, such as laser light, to a desired beam size. This beam size may dictate the size of the plume of microparticles ejected from the surface of the absorption material formed as a portion of the launch platform.

Although not explicitly shown in FIG. 8, the process for forming the treatment device may also include other acts such as providing a controller that is configured to control light energy emitted from the light source. For instance, the controller may control at least one of the pulse energy and pulse duration of the light energy in instances where the light source is a laser light source.

The method 800 discussed above in reference to FIG. 8 depicts one particular sequence of acts in a particular embodiment. Some acts are optional and, as such, may be omitted in accord with one or more embodiments. For instance, in some embodiments, the metal layer may be optional. Additionally, the order of acts can be altered, or other acts can be added, without departing from the scope of the embodiments described herein. For instance, the method may further comprise manufacturing or otherwise forming the microparticles. As discussed above, the microparticles may be formed from one or more liquid phases, and other formation techniques are also within the scope of this disclosure. Microparticles that contain polymers may be prepared using polymerization techniques. Pharmaceutical agents that comprise the microparticles may be attached to the microparticle or may form the microparticle itself. For instance, a microparticle may contain a core that includes a pharmaceutical agent and a shell that contains a drug delivery or drug carrying substance (or vice versa). In some embodiments, a biodegradable polymer may be used to form a portion of the microparticle and a radioisotope may be bound to the microparticle.

Having thus described several aspects of at least one example, it is to be appreciated that various alterations, modifications, and improvements will readily occur to those skilled in the art. For instance, examples disclosed herein may also be used in other contexts. Such alterations, modifications, and improvements are intended to be part of this disclosure, and are intended to be within the scope of the examples discussed herein. Accordingly, the foregoing description and drawings are by way of example only.

What is claimed is:

1. A system for treating a target condition, comprising:
a launch platform, comprising:
a substrate;
a layer of light absorption material deposited on the substrate; and
a layer of microparticles comprising at least one therapeutic agent deposited on the layer of absorption material;
a light source; and
a controller coupled to the light source and configured to control light energy emitted from the light source.

2. The system of claim 1, wherein the layer of microparticles is arranged as a self-assembled monolayer.

3. The system of claim 1, wherein the substrate is transparent to infrared light.

4. The system of claim 1, wherein the substrate further comprises a layer of metal deposited on the substrate between the substrate and the layer of light absorption material.

5. The system of claim 1, wherein the at least one therapeutic agent is a pharmaceutical or a radioactive material.

6. The system of claim 5, wherein the microparticles further comprise at least one of a metal, an imaging agent, a polymer, and a drug carrier.

7. The system of claim 1, wherein the light source is a laser light source.

8. The system of claim 7, wherein the controller is configured to control at least one of the laser pulse energy and pulse duration.

9. The system of claim 1, wherein the launch platform is configured to be interchangeable.

10. A device for treating a target condition, comprising:
a housing;
a launch platform positioned within the housing, the launch pad comprising:
a substrate;
a layer of metal deposited on a first surface of the substrate;
a layer of light absorption material deposited on the layer of metal; and
a layer of microparticles comprising at least one therapeutic agent deposited on the layer of light absorption material; and
a light source coupled to the housing and configured such that light energy emitted from the light source is in communication with a second surface of the substrate.

11. The device of claim 10, further comprising a focusing lens disposed between the light source and the second surface of the substrate, the focusing lens configured to focus light energy passing therethrough.

12. The device of claim 11, wherein the light source is configured as a laser light source coupled to an optical fiber such that a first end of the optical fiber is coupled to the laser light source and a second end of the optical fiber terminates in proximity to the focusing lens.

13. The device of claim 10, further comprising a controller in electrical communication with the light source and configured to control the light energy emitted from the light source.

14. The device of claim 10, wherein the launch platform is configured to be connected to the housing.

15. The device of claim 10, wherein the at least one therapeutic agent is a pharmaceutical or a radioactive material.

16. The device of claim 15, wherein the layer of metal is gold and the layer of absorption material is PDMS.

17. A method for forming a treatment device, comprising:
depositing a layer of metal onto a first surface of a substrate;
depositing a layer of light absorption material onto the layer of metal;
depositing a layer of microparticles comprising at least one therapeutic agent onto the layer of light absorption material; and
coupling a light source to a second surface of the substrate.

18. The method of claim 17, wherein the microparticles are deposited such that they form a self-assembled monolayer.

19. The method of claim 17, further comprising providing a controller that is configured to control light energy emitted from the light source.

20. The method of claim 17, further comprising positioning a focusing lens in between the light source and the second surface of the substrate.
